(19) United States
(12) Patent Application Publication
(10) Pub. No.: US 2007/0196414 A1
(43) Pub. Date: Aug. 23, 2007

(54) Device to prevent blood leakage using intravenous catheters

(76) Inventors: Ola Hammarsten, Kungsbacka (SE); Niklas Brywe, Saro (SE)

Correspondence Address: LYNN E BARBER, P O BOX 16528, FORT WORTH, TX, 76162, US

(21) Appl. No.: 11/360,271

(22) Filed: Feb. 23, 2006

(51) Int. Cl. A61F 13/00

(52) U.S. Cl. 424/422; 215/228; 604/167.01

(57) ABSTRACT

A device for preventing blood leakage during use of an intravenous catheter and needle using a stopper made of a material that is air permeable when dry; upon exposure to blood or other liquids bercomes rapidly liquid impermeable; is non-toxic and validated to use on humans; is inexpensive; is stable and withstands normal sterilization procedures such as heating and ionizing radiation; and is flexible and able to seal the hole when the needle is removed from the IV catheter during the cannulation process.

### BACKGROUND OF THE INVENTION

1. Field of the Invention

The present invention relates generally to blood leakage protection and other safety issues using intravenous (IV) catheters, used for administration of medicine to a patient's bloodstream or to allow introduction of instruments for intra luminal manipulation in blood vessels. The invention can also be used to prevent unwanted leakage during other types of catheterization procedures including but not limited to drainage of ascites, pleural and pericardial exudation.

2. Description of the Related Art

It can be appreciated that administration of medicine and liquids to the blood of patients is used frequently in hospital care. Intravenous and arterial infusion at hospitals requires a safe access to the patient's blood stream. For this purpose intravenous and arterial catheters are used. Intravenous and arterial catheters are devices with an introducer needle covered with a small tube that is forced into the vessel. The introducer needle is then withdrawn, leaving the tube inside the vessel, through which it is possible to administer medicine and other liquids to the patient. This tube can also be used to insert mechanical devices to do intra luminal manipulations such as removal of blood clots.

A common problem with conventional intravenous catheters is leakage of blood that occurs when the catheter is inserted into the vessel (cannulation process). The leakage of blood through the infusion channel of the device occurs as the health care worker is searching for the vein or artery after penetrating the skin with the needle. As the needle is inserted into the lumen of the vessel, blood will flow from the vessel into the catheter and thereby affirm the correct position of the catheter. The current forms of catheters allow the blood to flow freely through the infusion channel in the catheter and the only means to prevent blood leakage is to hold a pressure over the vessel , using for example the clinician's thumb or another mechanical method, which can be hard to do under stress, resulting in substantial soiling of blood during the cannulation procedure. This exposes the health-care workers to the risk of blood-borne infections and poses a significant working environment problem.

Another problem with intravenous and arterial catheters is needle stick injury from the introducer needle. Since this blood-soiled needle is withdrawn from the catheter and put aside during the process of preparing intravenous access, the needle that is contaminated with blood poses a substantial risk for needle stick injuries and blood borne infection. These infections are mainly HIV, hepatitis C and hepatitis B.

Since the mid-1980s, concern over the risk of accidental needle stick injuries and leakage of blood has spawned a number of design approaches for safety needle devices. Devices relating to needle stick protection can be broadly categorized as either sliding sheath needle devices, wherein a physical barrier is positioned over the needle tip after use or as devices with needle retraction, wherein the exposed portion of the needle is retracted into the device after use.

Patents directed to needle stick protection include U.S. Pat. No. 4,762,516 of Luther et al. The disclosure of this patent and all other patents and patent applications referred to herein is incorporated herein by reference. This patent describes an assembly of a needle catheter protector. The assembly comprises an elongated housing which mounts to the needle. A needle guard is slidably mounted within the housing and is adapted to be moved forward along the needle. Following use, the needle and housing are retracted and the needle guard permanently locks with the housing while it covers the needle.

U.S. Pat. No. 4,834,718 of McDonald describes an intravenous catheter apparatus. The catheter is introduced with the aid of a needle, which is thereafter withdrawn from the patient's vessel into a protective housing without exposing the needle during intermediate stages of the process. In particular, means are provided for latching the housing in place after the needle is withdrawn and for locking a catheter hub in place until the needle is withdrawn. Withdrawal and locking are affected in one continuous motion.

U.S. Pat. No 6,527,747 of Adams et al. describes an intravenous catheter apparatus with needle shield that is connected to the needle hub by a pleated tether. The tether prevents unwanted proximal movement of the needle with respect to the needle shield once the needle has been withdrawn into the needle shield.

U.S. Pat. No 6,689,102 of Greene discloses an intravenous catheter assembly including a catheter hub and a needle, which extends through and is withdrawn into the needle cover. A needle stop is attached to the needle and includes a two locking positions. The first locking position prevents the catheter hub from disengaging from the needle cover, and the second locking position allows the catheter hub to disengage from the needle cover.

U.S. Pat. Application Pub. No. 20050075606A1 of Botich et al. provides an automatic needle retraction mechanism in which needle retraction is effectuated by normal operation of inserting the catheter into the patient's vessel , without the need to perform a separate manual step.

Another example of a catheter having a safety mechanism is disclosed in U.S. Pat Application Pub. No. 2004/0078002. The catheter hub has a retainer for keeping the protector within the catheter hub until the catheter assembly and needle assembly are separated wherein a flange engages the groove and secures the protector to the needle.

U.S. Pat. No 6,709,419 of Woehr describes a needle shield, which is on the needle within the interior of the catheter hub and is biased axially within the catheter hub with its ends being fixed to prevent axial expansion. Other needle tip protectors disposed within the catheter hub are described in U.S. Pat. No 6,652,486 of Bialecki et al. and EP 1 421 969.

Several venous catheters on the market have devices that protect from needle injury. While these products may be suitable for the particular purpose to which they address, they still have the problem with blood leakage during the cannulation process.

Prevention of blood leakage during the cannulation process is a technical challenge since blood must be able to flow through the catheter to allow the health care worker to confirm that the needle is correctly inserted into the vessel. A simple stopper that prevents all flow in the system until the insertion needle is retracted will not work since this will prevent the health care worker to confirm that the needle is inside the vessel. Therefore, a system that prevents blood leakage during the cannulation procedure must be permeable to air to allow free flow of blood from the vessel into the device but become impermeable to blood when the blood reaches the proximal end of the catheter. The system must also provide a strong enough leakage prevention to withstand the pressures that are generated when the IV catheter is flushed with saline after the cannulation procedure. This technical challenge is significant.

A patent directed to leakage protection is U.S. Pat. No. 6,719,726 of Meng et al. This patent provides an intravenous catheter and introducer needle assembly that uses a septum to minimize blood leakage from the assembly. The septum preferably is hollow to minimize drag as the introducer needle is removed from the catheter. A further patent application directed to leakage protection is U.S. Pat Application Pub. No. US20010014787 of Toyokawa e al. This patent application describes a mechanism for preventing blood leakage by pressuring and closing the catheter. Both these solutions are technically complicated and therefore costly, and do not use such a simple device as the present invention to solve the problem of blood leakage.

In the device of the present invention suitable materials are used to solve the problem well and at a low cost. Also, the device may be used to protect from leakage on existing catheters with minimal modification of these catheters. The essence of the invention is to allow initial free passage of air through the device, and then upon contact with blood or other fluids the passage through the device closes. Suitable materials for this function of the device are for example super-absorbent polymers (SAPs), which are materials that have the ability to absorb and retain large volumes of water and aqueous solutions. This makes them ideal for use in water absorbing applications such as baby diapers (nappies) and adults incontinence pads to absorbent medical dressings and controlled release medium. Preferred materials for use in the interior of the invention include both early super-absorbents as well as more recently developed materials. The outside is preferably made of PVC, polyethylene, polypropene or SEBS (Styrene-Ethylene-Butylene-Styrene Based) Thermoplastic Elastomer.

Early super-absorbents were made from chemically modified starch and cellulose and other polymers like poly(vinyl alcohol)(PVA), poly(ethylene oxide)(PEO) all of which are hydrophilic and have a high affinity for water. When lightly cross-linked, chemically or physically, these polymers became water-swellable but not water-soluble.

Today's super-absorbent polymers are made from partially neutralized, lightly cross-linked poly(acrylic acid), which has been proven to give the best performance versus cost ratio. The polymers are manufactured at low solids levels for both quality and economic reasons, and are dried and milled into granular white solids. In water they swell to a rubbery gel that in some cases can be up to 99 wt % water. The hydroxyl group in poly(2-hydroxyethyl methacrylate)(PHEMA) can be derivatized to produce a wide range of polymeric reagents suited to pharmaceutical and chromatographic applications as well as to immobilize biopolymers, encapsulate mammalian cells and other sensitive compounds. PHEMA prepared by bulk polymerization forms gels by entrapping water or other liquids. Polymerization in the presence of a liquid diluent, below a critical concentration, leads to gels. On equilibrium swelling, PHEMA attains a three-dimensional rubbery state. In swollen cross linked gels, termed “fishnet gels”, the crosslinks are separated by large zones of flexible polymer strands, structurally akin to typical rubber-like networks. Cross linked PHEMA displays rubber-like behavior above the glass transition temperature (≈70-90° C.) or when swollen with water or polar diluents.

Other possible materials are, for example, carboxymethylcellulose (CMC; also called carboxymethyl cellulose) and methylcellulose, two of the most widely used water-soluble derivatives of cellulose, which have applications in the food, cosmetic and pharmaceutical industries. They are made by the reaction of alkali cellulose with methyl chloride or chloroacetic acid, respectively. The reaction conditions are usually chosen such that only partial substitution of the three-hydroxyl groups on each cellulose monomer are substituted. Thus cellulose derivatives such as methylcellulose and CMC are partially substituted derivatives of cellulose. They dissolve in water to form thick, aqueous solutions, the viscosity of which depends upon the concentration of the polymer and its molecular weight. Methylcellulose has been commercialized by several companies, one of which is Dow Chemical Company which sells the product under the name METHOCEL A™ brand products. CMC is sold among others by Aqualon under the generic name sodium CMC. CMC is an attractive substance in regard that it is non toxic and used as food supplement, known to be compatible with blood contact as it is used as gel forming substance in bum injury bandages and is very inexpensive.

U.S. Pat. Appl. Pub. No. 20030073663A1 (Wiseman et al.) describes bio-absorbable medical devices prepared by oxidizing derivates of cellulose including CMC. The resulting material is formed to devices particularly useful in limiting surgical adhesions, and for homeostasis, but do not mention the use of the materials in IC catheters to prevent blood leakage.

With respect to blood leakage protection, the device of the invention (the BloodLock System) herein substantially departs from the conventional concepts and designs of the prior art, and in so doing provides products primarily developed for the purpose of providing a blood free cannulation process using intravenous and arterial catheters, while preserving the health care worker's ability to affirm free passage to the vessel and in some versions of the invention also combine the leakage protection device with a needle protection effect.

### SUMMARY OF THE INVENTION

The invention herein provides an IV safety catheter and introducer needle assembly that prevents blood leakage from the assembly during the cannulation procedure, thereby preventing infection with blood borne infections such as HIV and hepatitis. The blood-free cannulation also protects from the discomfort and anxiety that is associated with blood contamination and therefore improves the working environment for health care workers.

Another object of this invention is to provide an IV catheter and introducer needle. assembly that uses the device of the invention to prevent blood leakage from the assembly.

It is still another object of this invention to provide an IV catheter and introducer needle assembly that uses the device of the invention containing CMC powder combined with foam-rubber or other porous support to prevent blood-leakage.

It is also an object of this invention to provide an IV catheter and introducer needle assembly that uses the device of the invention containing CMC or other forms of cellulose fibers that can be formed to a porous stopper, to prevent blood-leakage.

Another object of this invention is to provide an IV catheter and introducer needle assembly to prevent blood leakage from the assembly using the device of the invention containing hydrophilic plastic polymers such as neutral polyacrylic acid and other forms of super absorbent polymers.

A further object of this invention is to provide an IV catheter and introducer needle assembly to prevent blood-leakage using the device of the invention containing powder made from super absorbent polymers, in combination with foam rubber.

The device of the invention absorbs any fluid from flowing into or out of the proximal end of the catheter. It is transparent, so that the health-care worker can immediately and easily confirm proper placement of the catheter into the patient's blood vessel.

### BRIEF DESCRIPTION OF THE DRAWINGS

FIG. 1 is a schematic view of the side (A) and front (B) of the device of the invention.

FIG. 2 is a schematic view of the device of the invention adapted to an IV catheter VENFLOT PRO™ from Becton & Dickinson, Franklin Lakes, N.J. USA, with FIG. 2A showing the catheter hub and infusion port of this catheter; FIG. 2B showing the needle and needle holder; FIG. 2C showing the device; FIG. 2D showing the assembled device as used in this catheter; and FIG. 2E showing the needle inserted in the assembled device and catheter.

FIG. 3 is a schematic view of the device of the invention adapted to an IV catheter VENFLOT PRO™ from Becton & Dickinson, Franklin Lakes, N.J. USA, where the catheter hub 16 has been extended to allow room for the invention. FIG. 3A shows the catheter hub and infusion port of this catheter; FIG. 3B shows the needle and needle holder, FIG. 3C shows the device; FIG. 3D shows the assembled device as used in this catheter; and FIG. 3E shows the needle inserted in the assembled device and catheter.

FIG. 4 is a schematic view of the device of the invention adapted to an IV catheter with needle protection device, PROTECTIVE ACUVANCE™ from Medex, Inc. Carlsbad, CAlif. USA. FIG. 4A shows the catheter hub and infusion port of this catheter; FIG. 4B shows the needle and needle holder; FIG. 4C shows the device; FIG. 4D shows the assembled device as used in this catheter; and FIG. 4E shows the needle inserted in the assembled device and catheter.

FIG. 5 is a schematic view of the device of the invention adapted to an IV catheter VASOFIX™, from B. Braun Melsungen AG, Melsungen Germany. FIG. 5A shows the catheter hub and infusion port of this catheter; FIG. 5B shows the needle and needle holder; FIG. 5C shows the device; FIG. 5D shows the assembled device as used in this catheter; and FIG. 5E shows the needle inserted in the assembled device and catheter.

FIG. 6 is a schematic view of the device of the invention adapted to an IV catheter SURSHIELD VERSATUS-WP, BSN medical GmbH & Co, Hamburg, Germany. FIG. 6A shows the catheter hub and infusion port of this catheter; FIG. 6B shows the needle and needle holder; FIG. 6C shows the device; and FIG. 6D shows the needle inserted in the assembled device and catheter.

### DETAILED DESCRIPTION OF THE INVENTION AND PREFERRED EMBODIMENTS THEREOF

The device of the invention comprises a plastic stopper filled with a porous material that is converted to gel upon exposure to blood. The gel substance is not permeable and therefore stops all flow of blood out of the IV catheter during cannulation. The gel substance is strong enough to withstand the pressure that is generated when the IV catheter is flushed with saline after the cannulation procedure. When the IV catheter is washed of blood and closed, the device can be removed and replaced with an infusion line or with a stopper.

The gel-forming material in the device of the invention must have the following properties: 1) it should be air permeable when it is dry; 2) upon exposure to blood or other liquids the material should form a strong gel or by other transitions become liquid impermeable in less than 1 second (and preferably 0.5 seconds); 3) the material must be non-toxic and validated to use on humans since it will be in contact with the patient's blood during the cannulation procedure; 4) it is desirable that the material should be inexpensive; 5) the material should be stable and withstand normal sterilization procedures such as heating and ionizing radiation; and 6) the material should be flexible and able to seal the hole when the needle is removed from the IV catheter during the cannulation process.

The gel-forming time is dependent on the grain size in the powder. When the grain size is below 0.05 mm in diameter, the gelling time can be below 0.1 seconds. Foam rubber is permeable to air and flexible enough to expand and seal any hole generated by needles. Foam rubber saturated with carboxymethyl cellulose powder therefore fulfils all requirements to function in the device of the invention.

Thus, to be able to form a material that can be placed in the device of the invention using carboxymethyl cellulose, the powder must be combined with foam rubber or other porous support.

Another embodiment of the invention is to use fibrous carboxymethyl cellulose (Aquacel) or other forms of cellulose fibers that can be formed to a porous stopper. When this material is exposed to blood or other liquids it forms a strong gel that prevents further flow of liquids. It is also possible to use hydrophilic plastic polymers such as neutral polyacrylic acid and other forms of super absorbent polymers. Super absorbing polymers can be used to make fibers, foams or other porous materials that can be used in device. It is also possible to make powder from superabsorbent polymers that can be used in combination with foam rubber as described above.

The features of the present invention will be more clearly understood by reference to the following examples, which are not to be construed as limiting the invention.

### EXAMPLE 1

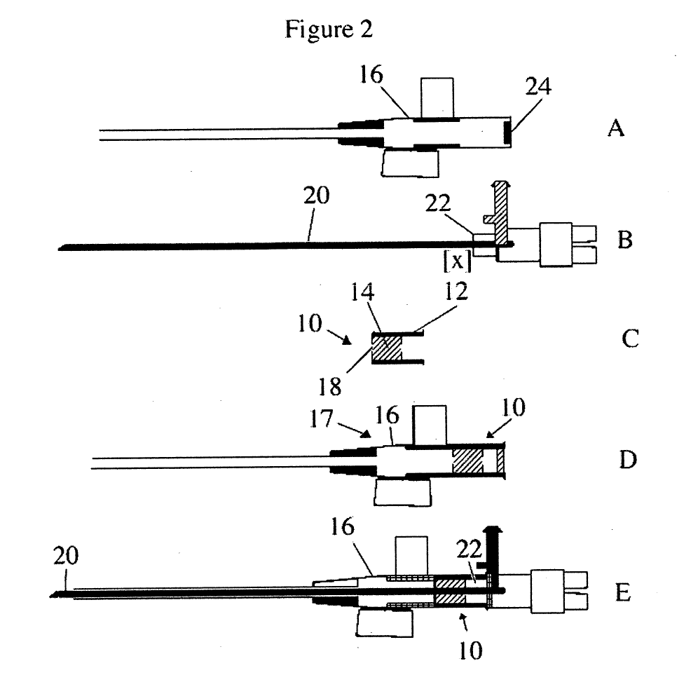

The device 10 of the invention in this example comprises a small plastic container 12, made by any means known in the art, for example, by plastic mold injection. The size of this container (“BloodLock™) depends on the specific needle for which it is to be used. Thus, for the version shown in FIG. 2 (Becton & Dickinson), it has a length of 9 mm, a diameter of 5 mm, with the forward hole having a diameter of 1.5 mm and the aft hole having a diameter of 4.5 mm.

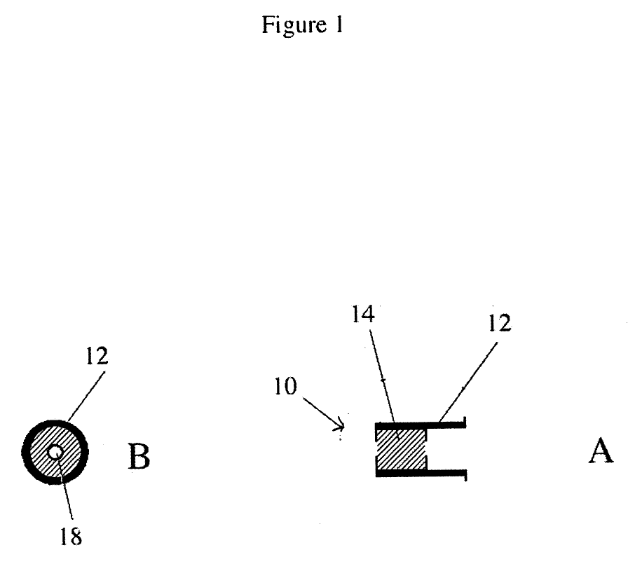

In FIG. 2 and thereafter, various versions of the invention are shown containing a carboxymethyl saturated foam rubber stopper 14 as shown in FIG. 1. FIG. 2A showing the catheter hub 16 and infusion port of this catheter 24, FIG. 2B showing the needle 20 and needle fixation holder 22, FIG. 2C showing the device 10. As shown in FIG. 2D, in the assembly of the invention 17, the device 10 fits into the infusion port 24 of, for example, a Venflon Pro (Becton & Dickinson, Franklin Lakes, N.J. USA) or a standard type of venflon and contains a channel 18 for the needle 20. To allow the stopper 10 to fit in the Venflon Pro, the needle fixation holder 22 known in the art has been shortened by 5 mm as shown at “X” in FIG. 2B to allow room for the device of the invention in the catheter hub 16 (FIG. 2A).

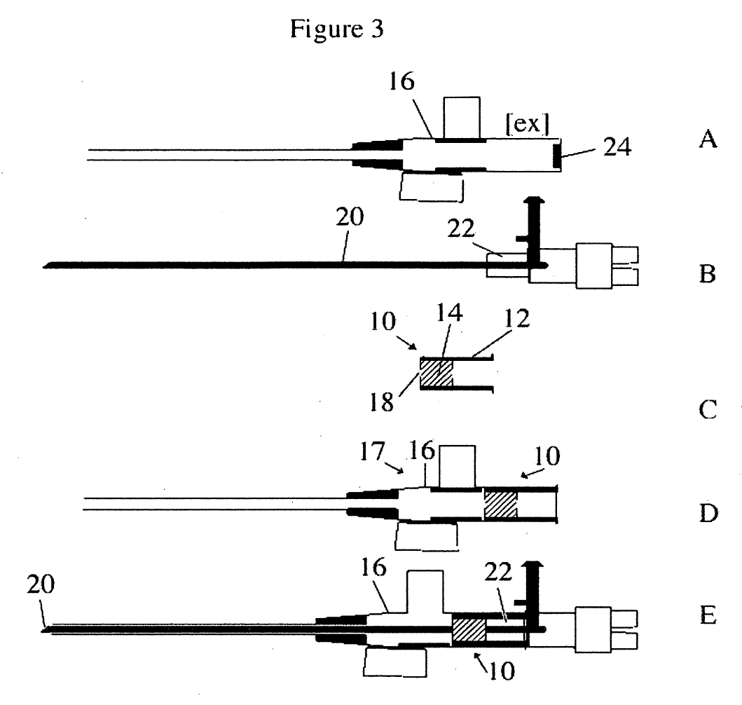

Alternatively, the catheter hub 16 may be extended (shown by “ex”) to allow the device to fit into the catheter as shown in FIG. 3A.

When Venflon Pro is assembled (e.g., FIG. 2E, 3E, etc.), the needle 20 is inserted through the channel 18, penetrating the carboxymethyl cellulose saturated foam rubber stopper 14. This design of the device can be adapted to all IV catheters with similar design as Venflon Pro.

During the cannulation process, when the needle is inserted into the vessel, blood flows through the catheter to the stopper 14. This allows the health care worker to affirm correct positioning in the vessel. The blood converts the carboxymethyl cellulose to a gel that prevents further blood flow. When the needle 20 is retracted the foam rubber expands to seal the needle puncture hole and prevent any leakage of blood. After the needle has been removed the Venflon Pro is flushed with saline until it is blood-free, as a part of the normal cannulation procedure. The strong gel that is formed from the carboxymethyl cellulose prevents any leakage during the flushing procedure. The device of the invention 10 can then be safely removed from the Venflon Pro without any leakage of blood and replaced with a stopper or an infusion line.

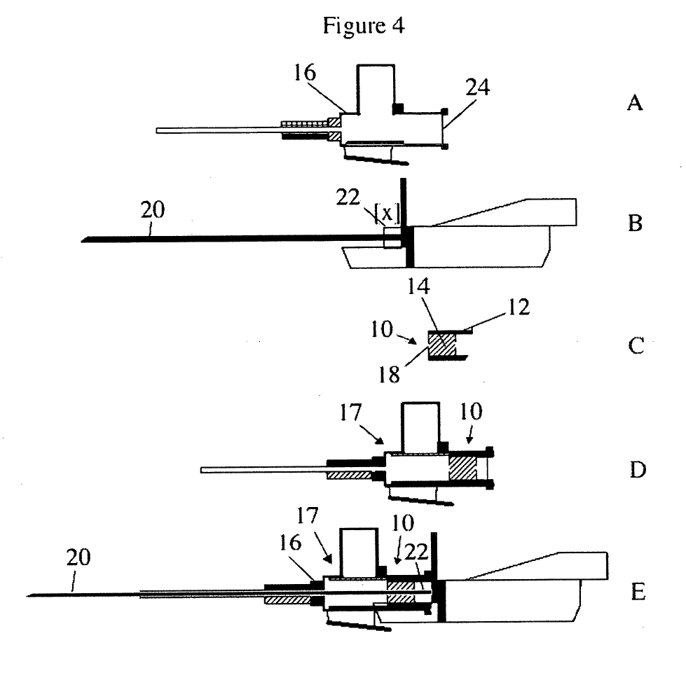

### EXAMPLE 2 (FIG. 4)

The device of the invention also fits into the infusion port 24 of Protective Acuvance 2 (FIG. 4D) and functions similarly as described for Venflon Pro (Example 1). The needle fixation point in the needle holder 22 has been shortened by 5 mm, at “X” to allow room for the invention in the catheter hub 16 with this needle (FIG. 4). Alternatively, the catheter hub 16 may be extended to allow the device to fit into the catheter the same way as shown in FIG. 3.

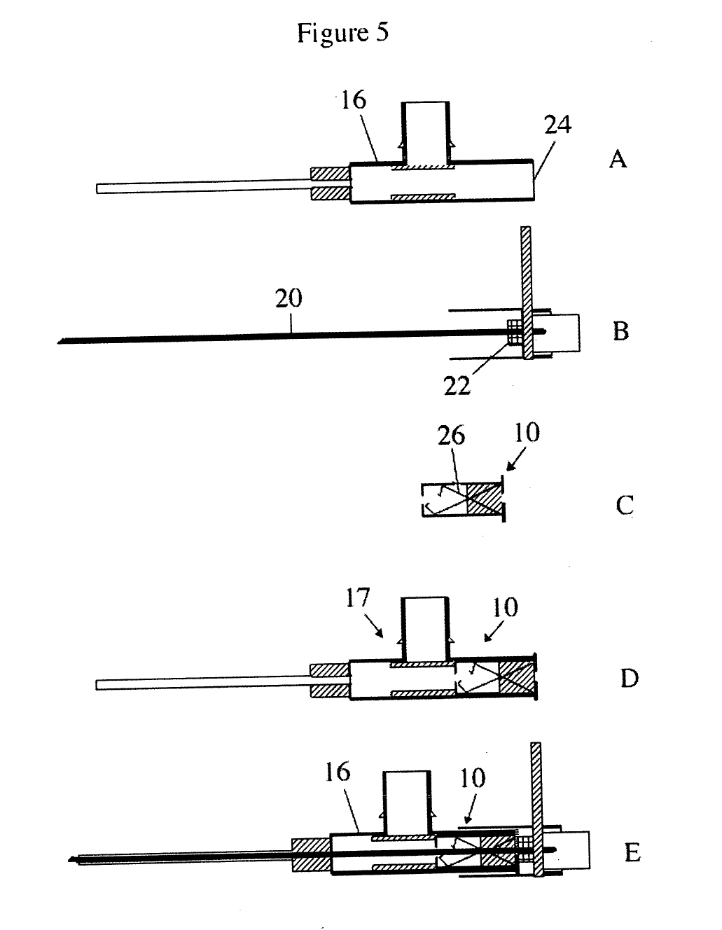

### EXAMPLE 3 (FIG. 5)

The device 10 of the invention has been adapted to fit the infusion port 24 of Vasofix Safety (Brown) and to enclose the spring 26 that functions as the needle protection device. The carboxymethyl cellulose saturated foam rubber stopper 14 has been placed in the proximal end of 10 to allow free movement of the spring. Therefore, the spring has been enclosed in the device so that both the needle protection and the prevention of leakage is contained in a single device as shown in C. To allow room for the invention in the catheter hub 16, the needle fixation point 22 have been shortened by 5 mm compared to the regular version of Vasofix safety for example at the time of manufacture of the version that will be fitted with the invention. Alternatively, the catheter hub 16 may be extended to allow the device to fit into the catheter similar to FIG. 3 (not shown).

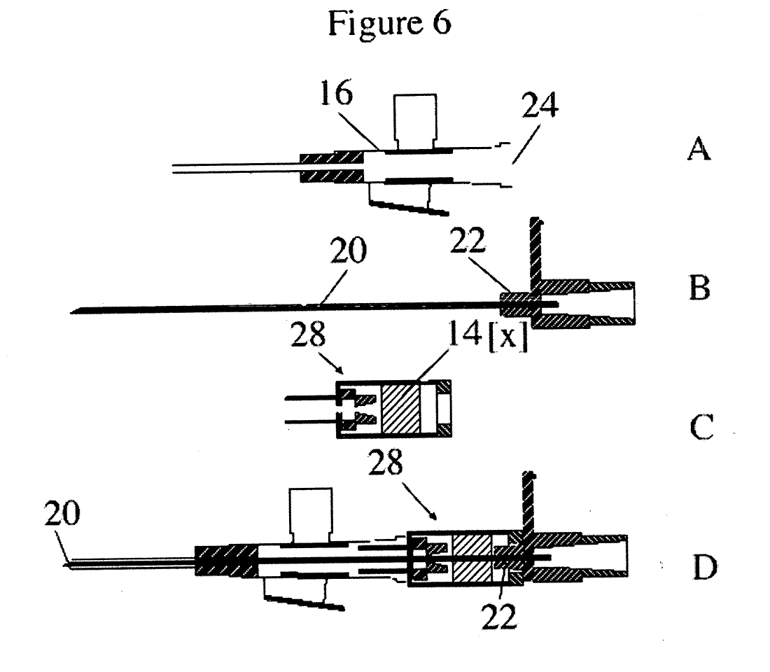

### EXAMPLE 4 (FIG. 6)

The protective function provided by Surshield Versatus-WP (Terumo) can be obtained by just adding a carboxymethyl cellulose saturated foam rubber stopper 14 in the needle protection device 28 as shown in FIG. 6C. To allow room for the stopper in the catheter hub 16 the needle fixation point 22 has been shortened by 5 mm at “X” for example at the time of manufacture of the version that will be fitted with the invention. Alternatively, the catheter hub 16 may be extended to allow the device to fit into the catheter similar to FIG. 3 (not shown).

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A device for preventing blood leakage during use of an intravenous catheter and needle, comprising a stopper having dimensions and shape to fit in the catheter and being filled with a porous material that is converted to gel upon exposure to blood.

2. The device of claim 1, wherein the porous material is: a) air permeable when dry; b) liquid impermeable in less than 1 second after exposure to liquid; c) non-toxic and validated to use on humans; d) inexpensive; e) stable and withstands normal sterilization procedures such as heating and ionizing radiation; and f) flexible and able to seal a hole when the needle is removed from the catheter during cannulation.

3. The device of claim 1, wherein the stopper is made from a substance selected from the group consisting of carboxymethyl cellulose and methylcellulose.

4. The device of claim 1, wherein the stopper is made of carboxymethyl cellulose and a porous support.

5. The device of claim 1, wherein the material is foam rubber saturated with carboxymethyl cellulose powder.

6. The device of claim 5, wherein the carboxymethyl cellulose has a grain size is below 0.05 mm.

7. The device of claim 1 wherein the stopper comprises cellulose fibers that can be formed to a porous stopper.

8. The device of claim 7, wherein the stopper comprises carboxymethyl cellulose.

9. The device of claim 1, wherein the stopper comprises a hydrophilic plastic polymer.

10. The device of claim 9 wherein the stopper comprises neutral polyacrylic acid.

11. The device of claim 1, wherein the stopper is made of powder from superabsorbent polymers in combination with foam rubber.

12. The device of claim 1, wherein the stopper is encased in a plastic container.
